(19) United States
(12) Patent Application Publication
(10) Pub. No.: US 2023/0405024 A1
(43) Pub. Date: Dec. 21, 2023

(54) NOVEL NANO-FORMULATION OF CANNABIDIOL (CBD) AND OTHER CANNABINOIDS FOR TREATMENT OF OCULAR DISORDERS

(71) Applicant: Michael Milane, Orange, CA (US)

(72) Inventor: Michael Milane, Orange, CA (US)

(21) Appl. No.: 17/911,230

(22) PCT Filed: Mar. 15, 2021
(86) PCT No.: PCT/US2021/022384

§ 371 Date: Sep. 13, 2022

Related U.S. Application Data
(60) Provisional application No. 62/989,309, filed on Mar. 13, 2020.

(51) Int. Cl. A61K 31/00; A61K 9/127

(52) U.S. Cl. CPC A61K 31/658; A61K 9/1272

(57) ABSTRACT

Compositions and methods are disclosed directed to nanoformulations of cannabidiol.

### CROSS REFERENCE TO RELATED APPLICATION

This application claims priority to U.S. Provisional Application 62/989,309, filed Mar. 13, 2020, which is incorporated herein by reference in its entirety.

### FIELD OF THE INVENTION

Disclosed herein are nano-formulations of cannabidiol (CBD) and other cannabinoids as well as methods of treating specific ocular diseases, disorders or conditions using the same. Also disclosed are methods of making such nanoformulations of cannabidiol.

### BACKGROUND OF THE INVENTION

Cannabidiol (CBD) is the major non-psychotropic constituent naturally present in Cannabis sativa L. plant isolated across the 1930s and 1940s, but chemically identified only in the 1960s by Mechoulam et al. [Mechoulam R, Shani A, Edery H, Grunfeld Y. Chemical basis of hashish activity. Science. 1970; 169:611-2.]. As well documented from Cannabis sativa L., it is also possible to extract over 100 different cannabinoids compounds considered as its most important bioactive constituents and mainly known for their psychoactive effects [Elsohly M A, Slade D. Chemical constituents of marijuana: the complex mixture of natural cannabinoids. Life Sci. 2005; 78:539-48.]. Among these the main studied is the Δ9-tetra-hydrocannabinol (Δ9-THC). This class of compounds have their effect mainly by interacting with specific receptors: the cannabinoid receptor type 1 (CB1), found on neurons and glial cells in various parts of the brain, and the cannabinoid receptor type 2 (CB2), found mainly in the body's immune system [Munro S, Thomas K L, Abu-Shaar M. Molecular characterization of a peripheral receptor for cannabinoids. Nature. 1993; 365:61-5.; Van Sickle M D, Duncan M, Kingsley P J, Mouihate A, Urbani P, Mackie K, et al. Identification and functional characterization of brainstem cannabinoid CB2 receptors. Science. 2005; 310:329-32.]. On the contrary, CBD has a very low affinity for these receptors (100 fold less than 49-THC) and when it binds it produces little to no psychoactive effects [Thomas A, Baillie G L, Phillips A M, Razdan R K, Ross R A, Pertwee R G. Cannabidiol displays unexpectedly high potency as an antagonist of CB1 and CB2 receptor agonists in vitro. Br J Pharmacol. 2007; 150:613-23.].

CBD is able to exert multiple pharmacological actions via CB1 and CB2 receptors involving intracellular pathways that play a key role in neuronal physiology [Pertwee R G, Howlett A C, Abood M E, Alexander S P, Di Marzo V, Elphick M R, et al. International Union of Basic and Clinical Pharmacology. LXXIX. Cannabinoid receptors and their ligands: beyond CB(1) and CB(2). Pharmacol Rev. 2010; 62: 588-631.; Zuardi A W. Cannabidiol: from an inactive cannabinoid to a drug with wide spectrum of action. Rev Bras Psiquiatr. 2008; 30: 271-80.]. In particular, many actions of CBD seem to be mediated by binding transient receptor potential vanilloid type 1 (TRPV1) [Costa B, Giagnoni G, Franke C, Trovato A E, Colleoni M. Vanilloid TRPV1 receptor mediates the antihyperalgesic effect of the nonpsychoactive cannabinoid, cannabidiol, in a rat model of acute inflammation. Br J Pharmacol. 2004; 143:247-50., G protein-coupled receptor 55 (GPR55); [Pertwee R G. GPR55: a new member of the cannabinoid receptor clan? Br J Pharmacol. 2007; 152:984-6.] and 5-hydroxytryptamine receptor subtype 1A (5-HT1A) [Russo E B, Burnett A, Hall B, Parker K K. Agonistic properties of cannabidiol at 5-HT1 a receptors. Neurochem Res. 2005; 30:1037-43.]. These additional and novel cannabinoid receptors (CB 1 and CB2) have been identified in CB1 and CB2-knockout mice and are expressed in both central and peripheral nervous system [Buckley N E. The peripheral cannabinoid receptor knockout mice: an update. Br J Pharmacol. 2008; 153:309-18., 12. Valverde O, Karsak M, Zimmer A. Analysis of the endocannabinoid system by using CB1 cannabinoid receptor knockout mice. [Valverde O, Karsak M, Zimmer A. Analysis of the endocannabinoid system by using CB1 cannabinoid receptor knockout mice. Handb Exp Pharmacol. 2005; 117-45.].

Moreover, CBD and few of the specific cannabinoids have proved to have several anti-inflammatory activities and regulates cell cycle and immune cells functions [Rieder S A, Chauhan A, Singh U, Nagarkatti M, Nagarkatti P. Cannabinoid-induced apoptosis in immune cells as a pathway to immunosuppression. Immunobiology. 2010; 215:598-605.]. CBD is able to suppress the production of a wide range of pro-inflammatory cytokines, such as tumor necrosis factor (TNF)-α and interleukin-1 beta (IL-1β), chemokines, growth factors, as well as inhibition of immune cell proliferation, activation, maturation, migration and antigen presentation [Jean-Gilles L, Gran B, Constantinescu C S. Interaction between cytokines, cannabinoids and the nervous system. Immunobiology. 2010; 215:606-10; Mechoulam R, Peters M, Murillo-Rodriguez E, Hanus L O. Cannabidiol—recent advances. Chem Biodivers. 2007; 4:1678-92.]. CBD shows also a potent action in inhibiting oxidative and nitrosative stress, modulating the expression of inducible nitric oxide synthase (iNOS) and nitro-tyrosine as well as reducing production of reactive oxygen species (ROS) [Iuvone T, Esposito G, De Filippis D, Scuderi C, Steardo L. Cannabidiol: a promising drug for neurodegenerative disorders? CNS Neurosci Ther. 2009; 15:65-75.].

The current clinical applications of CBD have been limited by sub-optimal formulations in terms of therapeutic index and/or adverse effects. As such, there remains a need for improved formulations of CBD for ocular use.

### SUMMARY OF THE INVENTION

Disclosed herein are nanoformulations of a cannabidiol (CBD), as well as associated methods including methods of treatment and methods of manufacture. Advantageously, the CBD nanoformulations disclosed herein have desirable bioavailability, efficacy and safety.

In one embodiment, a nanoformulation of CBD is disclosed, comprising cannabidiol and at least one fractionated oil (e.g., fractionated coconut oil) comprising medium-chain fatty acids having between 6 and 8 carbon items, wherein the at least one fractionated oil does not contain fatty acids having greater than 8 carbon atoms.

In a particular embodiment, the at least one fractionated oil does not contain fatty acids having greater than 6 carbon atoms.

In a particular embodiment, the nanoformulation further comprises one or more additional ingredients selected from the group consisting of anti-oxidants and penetration enhancers.

In another particular embodiment, the nanoformulation of CBD has a bioavailability greater than about 15%.

In another embodiment, a method of treating a subject in need thereof with the nanoformulation of cannabidiol is disclosed, comprising administering to the subject a therapeutically effective amount of the nanoformulation for treating an ocular disease or disorder.

In a particular embodiment, the ocular disease is an anterior ocular disease.

In another particular embodiment, the ocular disease is a posterior ocular disease.

In one embodiment, the ocular disease is a retinal disease. In a particular embodiment, the retinal disease is selected from the group consisting of neovascular age-related macular degeneration (AMD), diabetic macular edema (DMO), and retinal vascular occlusions (RVO).

In certain embodiments, the method results in a reduction in one or more symptoms of the ocular disease or disorder. The reduction may be, for example, about 10% or more relative to a control.

In other embodiments, the method results in a reduction in the progress of the ocular disease or disorder compared to a control. The reduction may be, for example, about 10% or more relative to a control.

In a further embodiment, a method of manufacturing the nanoformulation of cannabidiol is disclosed, comprising (i) providing the cannabidiol and at least one fractionated oil comprising medium-chain fatty acids having between 6 and 8 carbon items, wherein the at least one fractionated oil does not contain medium-chain fatty acids having greater than 8 carbon atoms; and (ii) processing the cannabidiol and at least one short-chained fractionated oil to provide a nanomaterial.

### DETAILED DESCRIPTION OF THE INVENTION

Disclosed here are novel nanoformulations of Cannabinoids and more particular, optimal barrier delivery formulations, for enhanced therapeutic penetration and bioavailability, as well as methods of use and manufacturing for such nanoformulations.

### I. Definitions

The term “amplitude,” as used herein, refers to the maximum variation occurring in an acoustic variable. It is measured in units of pressure: MPa (Mega Pascals).

The term “associated”, as used herein, refers non-covalent interaction between two entities, e.g., molecules, compounds or combinations thereof mediated by one or more of hydrophobic, electrostatic, and van der Walls interactions.

The term “bioavailability, as used herein, refers the rate and extent to which a drug reaches at the site of action. The evaluation of topical bioavailability involves quantification of the target tissue itself.

The term “cannabis”, as used herein, refers hereinafter to a genus of flowering plants that includes three different species, Cannabis sativa, Cannabis indica and Cannabis ruderalis.

The term “cannabis extract” or “cannabis oil” as used herein refers to a substance obtained by extracting a raw cannabis plant material (e.g., dried hemp, cannabis leaves), using a solvent, wherein the solvent has substantially been removed.

The term “cannabinoid”, as used herein, refers to a large and diverse class of chemical compounds that act on cannabinoid receptors on cells that repress neurotransmitter release in the brain. The most well-studied include tetrahydrocannabinol (THC), cannabidiol (CBD) and cannabinol (CBN). Other known cannabinoids include cannabigerol (CBG) cannabichromene (CBC), cannabicyclol (CBL), cannabivarin (CBV), tetrahydrocannabivarin (THCV); CBDV cannabidivarin (CBDV); cannabichromevarin (CBCV), cannabigerovarin (CBGV); cannabigerol monomethyl ether (CBGM); tetrahydrocannbinolic acid (THCA); cannabidiolic acid (CBDA) and isomers and enantiomers thereof.

The term “carriers”, as used herein, refers to a material suitable for topical drug administration. Carriers and vehicles useful herein include any such materials known in the art, which are nontoxic and do not interact with other components of the composition in a deleterious manner.

The term “combination therapy,” as used herein, refers to the administration of two or more therapeutic agents to treat a therapeutic condition or disorder described in the present disclosure. Such administration encompasses co-administration of these therapeutic agents in a substantially simultaneous manner or a sequential manner. In either case, the treatment regimen will provide beneficial effects of the drug combination in treating the conditions or disorders described herein.

The term “conjugated”, as used herein, refers to covalent or ionic interaction between two entities, e.g., molecules, compounds or combinations thereof.

The term “dose”, as used herein, refers to a specified quantity of a pharmaceutical agent provided in a single administration.

The term “dosage unit”, as used herein, refers a form in which a pharmaceutical agent is provided.

The term “effective amount”, as used herein, refers to the administration of an amount of a given compound that achieves the desired effect. An effective amount may be a therapeutically effective amount or a prophylactically effective amount.

The term “energy activation”, as used herein, means activation by an energy source that causes thermal or chemical activity. Energy activation may be by any energy source known in the art. Exemplary energy sources include a laser, ultrasound, acoustic source, flashlamp, ultraviolet light, an electromagnetic source, microwaves, or infrared light. An energy absorbing compound absorbs the energy and become thermally or chemically active.

The term “fatty acid”, as used herein, refers to a carboxylic acid consisting of a hydrocarbon chain and a terminal carboxyl group, especially any of those occurring as esters in fats and oils. Most naturally occurring fatty acids have an unbranched chain of an even number of carbon atoms, from 4 to 28.

The term “fractionated oil”, as used herein, refers to an oil that has been refined, for example, by removing the long-chain triglycerides, i.e., triglycerides having 14 or more carbons.

The term “highly purified cannabinoids”, as used herein, refers to cannabinoids that have been extracted from the cannabis plant and purified to the extent that other cannabinoids and non-cannabinoid components that are co-extracted with the cannabinoids have been removed, such that the highly purified cannabinoid is greater than or equal to 98% (w/w) pure.

The term “inhibit,” as used herein, refers to prohibiting, preventing, restraining, and lowering, stopping, or reversing progression or severity, and such action on a resultant symptom.

The term “lipid”, as used herein, refers to a group of organic compounds that are esters of fatty acids and are characterized by being insoluble in water but soluble in many organic solvents. Lipids may be classified as: (1) “simple lipids” which include fats and oils as well as waxes; (2) “compound lipids” which include phospholipids and glycolipids; and (3) “derived lipids” such as steroids.

The term “lipid drug conjugate” or “lipoidal prodrug” as used herein refers to a drug covalently bound to a lipid, such as a fatty acid or phospholipid. The bond may be, for example, an ester bond, an amide bond, a disulfide bond or a hydrazine bone. Optionally, a linker or space may be used.

The term “lipid encapsulated”, as used herein, refers to a lipid nanoparticle that provides an active agent or therapeutic agent, with full encapsulation, partial encapsulation, or both.

The term “lipid nanoparticles” or “LPN”, as used herein, refers to lipid-based particles in the submicron range. Lipid nanoparticles can have structural characteristics of liposomes and/or have alternative non-bilayer types of structures. Lipid nanoparticles may comprise one or more lipid species.

The term “liposome”, as used herein, refers to a spherical vesicle of a lamellar phase of the lipid bilayer.

The term “local delivery”, as used herein, refers to tissue specific delivery or distribution.

The term “long-chain fatty acid”, as used herein, refers to a fatty acid containing 14 or more carbon atoms. For example, myristic acid (C14), palmitic acid (C16), stearic acid (C18), arachidic acid (C20) and the like.

The term “medium-chain fatty acid”, as used herein, refers to triglycerides containing fatty acids with between 6 and 12 carbon atoms. For example, caproic acid (C6), caprylic acid (C8), capric acid (C10) and lauric acid (C12).

The term “nanoemulsion”, as used herein, refers to a transparent, monophasic, optically isotropic and kinetically stable colloidal dispersions composed of oil, water, surfactant and cosurfactant with droplet sizes less than 100 nm.

The term “nanoparticle,” as used herein, refers to a particle having a diameter, such as an average diameter, from about 10 nm up to but not including about 1 micron, preferably from 100 nm to about 1 micron. The particles can have any shape. Nanoparticles having a spherical shape are generally referred to as “nanospheres”.

The term “nanostructured lipid carriers” or “NLC”, as used herein, refers to a colloid system composed of a fluid lipid phase embedded into a solid lipid matrix or localized at the surface of solid platelets and the surfactant layer.

The term “ocular disorder” or “ocular disease”, as used herein, refers to a disease, ailment or condition which affects or involves the eye, including the eyeball, including the cornea, and other tissues and fluids which constitute the eyeball, the periocular muscles (such as the oblique and rectus muscles) and the portion of the optic nerve which is within or adjacent to the eyeball.

The term “ocular region” or “ocular site” means any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

The term “ocular surface”, as used herein, refers to the wet-surfaced and glandular epithelia of the cornea, conjunctiva, lacrimal gland, accessory lacrimal glands, nasolacrimal duct and meibomian gland, and their apical and basal matrices, puncta and adjacent or related structures, including the eyelids linked as a functional system by both continuity of epithelia, by innervation, and the endocrine and immune systems.

The term “onset of action,” as used herein, refers to is the duration of time it takes for a drug's effects to come to prominence upon administration.

The term “ophthalmically acceptable”, as used herein, with respect to a formulation, composition or ingredient herein means having no persistent detrimental effect on the treated eye or the functioning thereof, or on the general health of the subject being treated, baring transient effects such as minor irritation or a “stinging” sensation.

The term “penetration enhancer”, as used herein, an agent or a combination of agents that improves the transport of molecules such as a pharmaceutically or cosmetically active agent into or through a natural membrane.

The term “phytocannabinoids”, as used herein, refers to cannabinoids that originate from nature and can be found in the cannabis plant. The phytocannabinoids can be isolated from plants to produce a highly purified extract or can be reproduced synthetically.

The term “plant oil”, as referred to herein, refers to oils derived from plants as opposed to petroleum or animals. They are triglycerides and contain various fatty acids. Most plant oils are liquid at room temperature.

The term “solid lipid nanoparticle” or “SLN”, as used herein, refers to a nanoparticle composed of lipids that are solid at room temperature with a surface covering of surfactant to stabilize them as a nano-dispersion.

The term “sonication”, as used herein, refers to a subset of mechanical vibration wherein sonic energy generated using a transducer or a probe or other mechanism capable of generating the desired frequency at the desired power, is transmitted to a material. The frequency of such sonic energy may be from 10 KHz to as much as 10 MHz. In this disclosure, when referring to sonication at frequencies less than 20 KHz it is understood that such frequencies are not technically ultrasound as they are in the audible range. As used herein, the term “ultrasonication” refers to sonication using a frequency or frequencies in the inaudible frequency range above about 20 KHz, generally from about 20 KHz to about 1 MHz. As those skilled in the art will appreciate, ultrasonication comprises the transmission of ultrasound energy.

The term “stable” and “stability” are used herein with reference to the shelf-life of a pharmaceutical product, and are related to the physical change, degradation or chemical decomposition of active pharmaceutical ingredients, which limits the shelf-life of a product. Each active pharmaceutical ingredient has its intrinsic stability, its degradation pathways and degradation products, in part depending on the formulation of which it is part, and the storage conditions. The major mechanisms of chemical degradation include oxidation, hydrolysis/dehydration, isomerization/epimerization, decarboxylation, dimerization/polymerization, photolysis and rearrangements. If a product is termed to be “stable” it means in this context that it can be stored for a prescribed time without any of these mechanisms advancing to the extent that compromises product efficacy and safety.

The term “subject, as used herein, refers to a human or non-human animal selected for treatment or therapy.

The term “synthetic cannabinoids”, as used herein, refers compounds that have a cannabinoid or cannabinoid-like structure and are manufactured using chemical means rather than by the plant.

The term “top down”, as used herein, refers to methods of structuring nanomaterial beginning with a bulk solid and obtaining a nanomaterial by structural decomposition. Top down is in contrast to precipitation (bottom up) techniques. Non-limiting examples include high pre

The terms “treat”, “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. For example, treatment of an ocular disease includes, but is not limited to, elimination of lowing or preventing the onset of the ocular disease, reducing the risk of the ocular disease, or arresting the development of the ocular disease.

The term “therapeutically sufficient flux”, as used herein, refers to permeation flux of the selected drug that delivers sufficient amount of drug to be clinically beneficial. “Clinically beneficial,” when referring to flux, means that at least a portion of the patient population can obtain some degree of benefit from the drug flux.

The term “ultrasound”, as used herein, refers to acoustic radiation with a frequency greater than about 20 kHz, e.g. about 50 kHz, 100 kHz, 500 kHz, 1,000 kHz, 5,000 kHz, 10,000 kHz, or greater. The ultrasound can be medical ultrasound, e.g. about 500 kHz to 30,000 kHz, about 1,000 kHz to 20,000 kHz, about 2,000 kHz to 15,000 kHz, or about 3,000 kHz to 10,000 kHz.

### II. Nanoformulation

Disclosed herein are nanotechnology-based formulations (nanoformulations) of CBD having improved properties relative to CBD formulations known in the art.

Cannabis extracts are hydrophobic (incompatible with water) and, as such, difficult to deliver to the water-based bloodstream. When consumed orally, for example, they undergo a slow process of gastrointestinal absorption, leading to a delayed onset of action (e.g., from 30 minutes to 2 hours), as well as a low (10-15%) and unpredictable bioavailability. Currently, most formulations of CBD are limiting in therapeutic efficacy, since most are at a sub-therapeutic dose, secondary to concentration, formulation, purity or delivery method.

The current technology and formulation provides nanoformulations of CBD having improved therapeutic efficacy, while still within the safety standards. In certain embodiments, the nanoformulation significantly enhances the bioavailability of CBD when administered by any route. In particular embodiments, the nanoformulations is characterized by an improved onset of action, and higher levels of therapeutic index. In one embodiment, this activity is increased by a factor 4-6×.

In a particular embodiment, the cannaboid is cannabidiol (CBD).

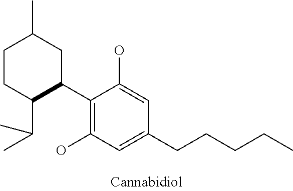

Cannabidiol is one of more than 120 cannabinoids identified in cannabis (marijuana), accounting for up to 40% of the plant's extract. Unlike the main psychoactive cannabinoid in marijuana, tetrahydrocannabinol (THC), CBD does not produce euphoria or intoxication. Cannabidiol has low affinity for the cannabinoid receptors CB1 and CB2 but is believed to act an indirect antagonist thereof. It is generally understood to be safe for human use. CBD is insoluble water but soluble in organic solvents, such as oil.

Cannabidiol from any source is suitable for use.

In a particular embodiment, the nanoformulation contains synthetic or semi-synthetic cannabidiol (e.g., chemically synthesized cannabidiol).

In a particular embodiment, the nanoformulation contains recombination cannabidiol (e.g., cannabidiol produced in yeast or another suitable host).

In a particular embodiment, the nanoformulation contains phytogenic cannabidiol, e.g., cannabidiol derived from plants, such as Cannabis sativa and Cannabis indica.

In one embodiment, the phytogenic cannabidiol is derived from a cannabis cultivar that is CBD dominant, i.e., produces higher levels of CBD (and/or CBDA) than THC (and/or THCA).

In another embodiment, the phytogenic cannabidiol derived from a cannabis cultivar that is CBD-rich.

In certain embodiments, the cannabis cultivar is characterized by greater than about 10%, greater than about 15%, greater than about 20%, greater than about 25%, greater than about 30%, greater than about 35%, greater than about 40%, greater than about 45% or greater than about 50% or more CBD.

In other embodiments, the cannabis cultivar is characterized by less than about 1.0%, less than about 0.5% or less than about 0.3% THC.

In certain embodiments, the cannabis cultivar is characterized by a CBD:THC ratio of greater than about 20:1, greater than about 25:1 or greater than about 30:1.

In another particular embodiment, the nanoformulation contains synthetic cannabidiol.

In another embodiment, the cannaboid is cannabinol, cannabichromine or cannabigerol.

In a particular embodiment, the nanoformulation further comprises at least one additional active agent, i.e., an active agent other than cannabidiol. In one embodiment, the additional active agent is a cannabinoid other than cannabidiol. The additional active agent may be, without limitation, a combination of any of the following cannabinoids:
THC (tetrahydrocannabinol, THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran), or others as identified for combination.

### Main Classes of Natural Cannabinoids

| Type | Skeleton | Cyclization |
|---|---|---|
| Cannabigerol-type CBG | 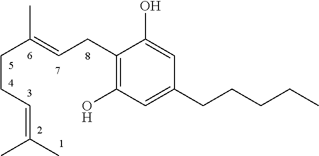 [SMILES: CCCCCc1cc(O)c(C/C=C(\C)CCC=C(C)C)c(O)c1] | 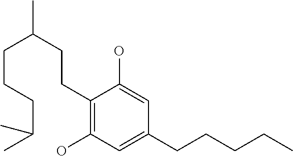 [SMILES: CCCCCc1cc(O)c(CCC(C)CCCC(C)C)c(O)c1] |
| Cannabichromene-type CBC | 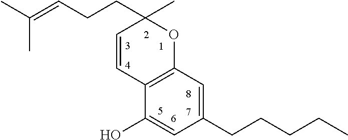 [SMILES: CCCCCc1cc(O)c2c(c1)OC(C)(CCC=C(C)C)C=C2] | 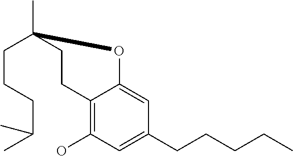 |
| Cannabidiol-type CBD | 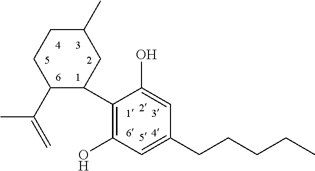 [SMILES: C=C(C)C1CCC(C)CC1c1c(O)cc(CCCCC)cc1O] | 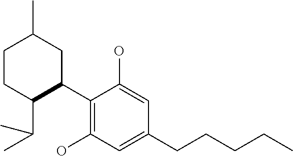 |
| Tetrahydrocannabinol- and Cannabinol-type THC, CBN | 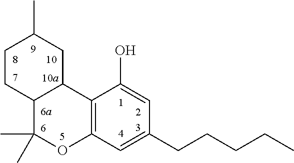 [SMILES: CCCCCc1cc(O)c2c(c1)OC(C)(C)C1CCC(C)CC21] | 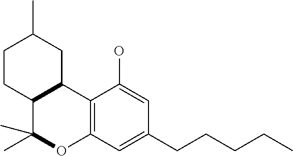 |
| Cannabielsoin-type CBE | 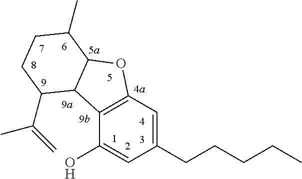 [SMILES: C=C(C)C1CCC(C)C2Oc3cc(CCCCC)cc(O)c3C12] | 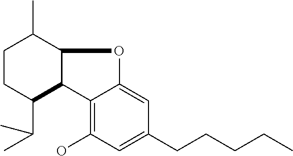 |
| iso- Tetrahydrocannabinol- type iso-THC | 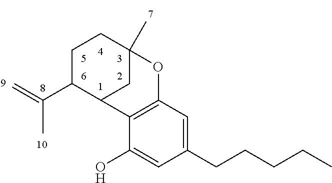 [SMILES: C=C(C)C1CCC2(C)CC1c1c(O)cc(CCCCC)cc1O2] | 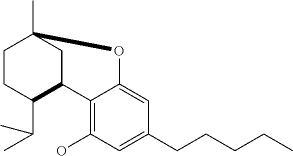 [SMILES: CCCCCc1cc(O)c2c(c1)O[C@]1(C)CC[C@H](C(C)C)C2C1] |
| Cannabicyclol-type CBL | 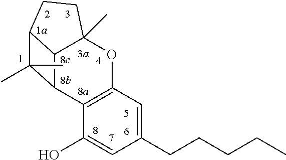 | 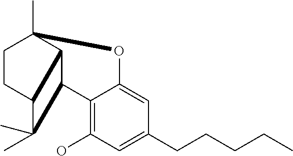 |
| Cannabicitran-type CBT | 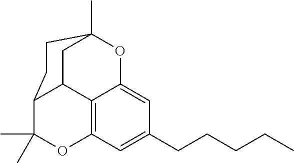 [SMILES: CCCCCc1cc2c3c(c1)OC(C)(C)C1CCC(C)(CC31)O2] | 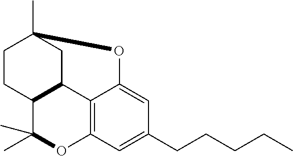 |

In certain embodiments, the therapeutic effect of the cannabidiol and the at least one additional active agent (e.g., the cannabinoid other than cannabidiol) is synergistic.

In one embodiment, the nanoformulation comprises a nanomaterial, such as a nanocarrier or nanoparticle. The size of the nanomaterial may vary. In one embodiment, the nanomaterial is between about 40 nm and about 100 nm, more particularly, about 50 nm and about 100 nm, more particularly, about 45 nm and about 55 nm.

In a particular embodiment, the nanomaterial is about 40 nm, about 45 nm, about 50 nm, about 55 nm, about 60 nm, about 65 nm, about 70 nm, about 75 nm, about 80 nm, about 85 nm, about 90 nm, about 95 nm or about 100 nm.

Cannabidiol may be associated with the nanomaterial is any suitable manner. In one embodiment, the cannabidiol is adsorbed or covalently attached to the nanocarrier's surface. In another embodiment, the cannabidiol is encapsulated within the nanomaterial, either completely or partially.

The nanocarrier may be, for example, organic (e.g., polymers, lipids), inorganic (e.g., metals, metal oxides) carbon-based.

In a particular embodiment, the nanocarrier is organic and more particularly, a lipid-based nanoparticle (LPN). The term “lipid-based”, as used herein, refers to compositions that primarily comprise lipids.

The lipid-based nanoparticle may vary in size. In a particular embodiment, the lipid-based nanoparticle has a mean particle size of between about 40 nm and about 100 nm, more particularly, about 50 nm and about 100 nm, even more particularly, about 45 nm and about 55 nm. In one embodiment, the lipid-based nanoparticle has a narrow polydispersity index (PI), e.g., lower than 0.2.

In a particular embodiment, the lipid-based nanoparticle is a liposome, micelle, flexible vesicle, lipoplex nanoparticle, lipid-drug conjugate (LDC), lipid nanocapsule (LNC), solid lipid nanoparticle (SLN) or nanostructured lipid carrier (NLC). In one embodiment, the cannabidiol is lipid-encapsulated. Alternatively, the lipid-based nanoparticle is nanoemulsion.

In another particular embodiment, the lipid-based nanoparticle is a nanostructured lipid carrier (NLC). In NLCs, the lipidic phase contains both solid (fat) and liquid (oil) lipids at ambient temperature. Compared to SLNs, NLCs possess lower melting point due to their oil content, while maintaining their particulate character and being solid at body temperature. In one embodiment, the NLC may be a Class I (imperfect type), class II (formless type) or class III (multiple type).

Any suitable lipid may be used. The lipid may be synthetic, semi-synthetic or a naturally-occurring lipid.

In one embodiment, the lipid is at least one fractionated oil. Fractionated oils containing fatty acids having between 4 and 8 carbon atoms, and more particularly, between 6 and 8 carbon atoms, have been determined to be advantageous to the oils having fatty acids with more than 8 carbon atoms. Fatty acids having between 6 and 8 carbon atoms are generally referred to as medium-chain fatty acids (MCFA). Fatty acids having more than 12 carbon atoms are generally referred to as long-chain fatty acids (LCFA).

Butyric (butanoic acid): CH₃(CH₂)₂COOH is a saturated fatty acid having 4 carbon atoms (C4:0); caproic (hexanoic acid): CH₃(CH₂)₄COOH is a saturated fatty acid having 6 carbon atoms (C6:0); caprylic (octanoic acid): CH₃(CH₂)6COOH is a saturated fatty acid having 8 carbon atoms (C8:0).

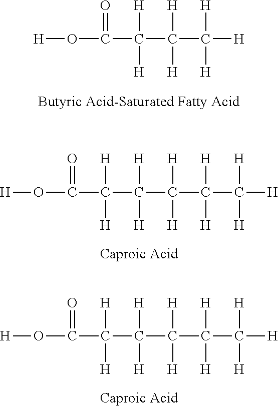 [SMILES: CCCC(=O)O ; CCCCCC(=O)O ; CCCCCC(=O)O]

Capric (decanoic acid): CH₃(CH₂)₈COOH is a saturated fatty acid having 10 carbon atoms (C10:0); auric (dodecanoic acid): CH₃(CH₂)₁₀COOH is a saturated fatty acid having 12 carbon atoms (C12:0); myristic (tetradecanoic acid): CH₃(CH₂)₁₂COOH is a saturated fatty acid having 14 carbon atoms (C14:0); palmitic (hexadecanoic acid): CH₃(CH₂)₁₄COOH is a fatty acid having 16 carbon atoms (C16:0); stearic (octadecanoic acid): CH₃(CH₂)₁₆COOH is a fatty acid having 18 carbon atoms (C18:0); arachidic (eicosanoic acid): CH₃(CH₂)₁₈COOH is a fatty acid having 20 carbon atoms (C20:0); behenic (docosanoic acid): CH₃(CH₂)₂₀COOH or C22:0

In a particular embodiment, the fractionated oil is enriched for fatty acids containing fatty acids having between 4 and 8 carbon atoms, and more particularly, between 4 and 6 carbon atoms or between 6 and 8 carbon atoms.

In a further particular embodiment, the fractionated oil is enriched for fatty acids having 6 carbon atoms.

In a particular embodiment, the fractionated oil contains fatty acids, wherein more than 80%, more than 85%, more than 90% or more than 95% of the fatty acids having between 4 and 8 carbon atoms, and more particularly, between 4 and 6 carbon atoms or between 6 and 8 carbon atoms.

In another particular embodiment, the fractionated oil does not contain any fatty acids having greater than 4 carbon atoms, more particularly, greater than 6 carbon atoms, or even more particularly, greater than 8 carbon atoms.

In one embodiment, the fractionated oil is selected from the group consisting of fractionated coconut oil, fractionated palm oil, fractionated palm kernel oil, fractionated sesame oil, fractionated soybean oil, fractionated almond oil, fractionated rapeseed oil, fractionated corn oil, fractionated sunflower oil, fractionated peanut oil, fractionated olive oil, fractionated castor oil, fractionated soybean oil, fractionated safflower oil, fractionated cottonseed oil, and combinations thereof.

In one embodiment, the nanocarrier comprises fractionated coconut oil. In a particular embodiment, the fractionated coconut oil is enriched for fatty acids having between 4 and 8 carbon atoms and more particularly, between 4 and 6 carbon atoms or between 6 and 8 carbon atoms. In one embodiment, the carrier is fractionated coconut oil having greater than 80%, greater than 85%, greater than 90%, or greater than 95% fatty acids having between 4 and 8 carbon atoms and more particularly, between 6 and 8 carbon atoms. In one embodiment, the carrier is fractionated coconut oil containing butyric, caprioic, caprylic fatty acids, or a combination thereof.

In certain embodiments, the nanocarrier comprises fractionated coconut oil containing greater than 80%, greater than 85%, greater than 90%, or greater than 95% butyric fatty acids.

In certain embodiments, the nanocarrier comprises fractionated coconut oil containing greater than 80%, greater than 85%, greater than 90%, or greater than 95% caprioic fatty acids.

In certain embodiments, the nanocarrier comprises fractionated coconut oil containing greater than 80%, greater than 85%, greater than 90%, or greater than 95% caprylic fatty acids

Fractionated coconut oil is a liquid at room temperature.

In a particular embodiment the nanoformulations contains two or more fractionated oils, e.g., fractionated coconut oil and fractionated palm oil.

The nanoformulation may further comprise one or more additional ingredients. These ingredients may include, for example, anti-oxidants, penetration enhancers, moisturizers, emulsifiers, gelling agents, surfactants, stabilizers, viscosity modifiers, antimicrobial preservatives, irritant-reducing additives, topical anesthetics or combinations thereof.

In one embodiment, the anti-oxidant is selected from the group consisting of butylated hydroxyltoluene (“BHT”), butylated hydroxyl anisole (“BHA”), alpha-tocopherol (Vitamin E), ascorbyl palmitate, ascorbic acid, sodium ascorbate, ethylenediamino tetraacetic acid, cysteine hydrochloride, citric acid, sodium citrate, sodium bisulfate, sodium metabisulfite, lecithin, propyl gallate, sodium sulfate, tert-butylhydroquinone (“TBHQ”) and combinations thereof In a preferred embodiment, the formulations contain alpha-tocopherol (Vitamin E), ascorbyl palmitate, or combinations thereof.

In one embodiment, the penetration enhancer is selected from the group consisting of propylene carbonate, transcutol, pyrrolidones such as N-methylpyrrolidone or N-hydroxyalkylpyrrolidone, azone, menthol, eucalyptol, nicotinamide, glycerol, mono- di- or polyglycols, ethylacetate or Eugenol.

In one embodiment, the moisturizer is selected from the group consisting of glycerin, sorbitol, polyethylene glycols, urea, and propylene glycol.

In one embodiment, the emulsifier is selected from the group consisting of acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

In one embodiment, the stabilizer is selected from the group consisting of albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and combinations thereof.

In addition to additional non-active components, the CBD nanoformulation may contain, in some embodiments, other active materials, such as drugs or agents conventionally used in the treatment of ocular diseases, disorders or conditions. Representative, non-limiting agents include antimicrobial agents, anti-inflammatory agents, anti-aging agents and the like.

The nanoformulation may be prepared for administration via various miscellaneous routes, for example, topical administration, transdermal administration, mucosal administration (intranasal, vaginal, etc.) and/or via inhalation.

These nanoformulations can be used in the preparation of individual, single unit dosage form.

The nanoformulation may be prepared for any mode of administration, including oral, sublingual, topical, nasal, rectal, vaginal, parenteral, ophthalmic, otic or the like.

Topical formulations may take the form of an oil, ointment, cream, lotion, patch, balm, salve, liniment, mouse, foam, bar, pencil, emulsion, gel or the like. The nanoformulation can also be incorporated in solid supports selected from the group consisting of hydrogels, wipes, patches and facial masks.

The amount of cannabidiol present in a nanoformulation of the present invention will be an amount effective to treat a given ocular condition based on observational studies or to prevent the same. In certain embodiments, the amount or concentration of cannabidiol is at least about 0.5% to 1% by weight based on the total weight of the nanoformulation.

In a particular embodiment, the amount or concentration of cannabidiol is between about and about 20%, about 1% and about 10% or about 2% to about 5% by total weight of the formulation.

In another particular embodiment, the amount or concentration of cannabidiol is about about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10% or more by weight based on the total weight of the nanoformulation.

The nanoformulation disclosed herein is stable for about two (2) years or greater than about 2 years.

Kits that include the nanoformulation disclosed herein are also contemplated. In certain embodiments, the nanoformulation (or components thereof) is comprised in a container. The container can be a bottle, dispenser, or package. The container can dispense a pre-determined amount of the nanoformulation. The container can include indicia on its surface. The indicia can be a word, an abbreviation, a picture, or a symbol.

### III. Methods of Use

Disclosed herein are methods of using the nanoformulation of CBD disclosed herein, such as methods of treating an ocular disease, disorder or condition. Currently, most formulation of CBD are limiting in therapeutic efficacy, since most are at a sub-therapeutic dose, secondary to concentration, formulation, purity or delivery method. The current technology and formulation aims to change these factors to optimize the delivery and concentration for fast acting efficacy, while still within the safety standards. In certain embodiments, the nanoformulation disclosed herein reduces side effects relative to CBD formulations known in the art.

In one embodiment, the method comprising administering a therapeutically effective amount of the CBD nanoformulation disclosed herein to a subject in need thereof, thereby treating an ocular disease, disorder or condition. In some embodiments, the method results in treating, preventing, or reducing the rate of pathogenesis of an ocular disorder.

The most suitable route of administration will depend on the nature and severity of the condition being treated. In one embodiment, the administration is ophthalmic. In a particular embodiment, the composition is administered to the ocular surface (topical). In another embodiment, administration is local (e.g., subconjunctival, intravitreal, retrobulbar, intracameral). In a further embodiment, administration is systemic.

In one embodiment, administration of the composition disclosed herein to a subject in need thereof comprises administration to the ocular surface using mechanical delivery means, i.e., dropping, spraying, bathing of the eyes in a solution, or a combination of these.

The methods disclosed herein are suitable for use in treating any ocular disease or disorder. The structure of eye can be divided into two main parts: anterior segment and posterior segment. Anterior segment of the eye occupies approximately one-third while the remaining portion is occupied by the posterior segment. Tissues such as cornea, conjunctiva, aqueous humor, iris, ciliary body and lens make up the anterior portion. Back of the eye or posterior segment of the eye include sclera, choroid, retinal pigment epithelium, neural retina, optic nerve and vitreous humor.

In a particular embodiment, the ocular disease or disorder treated using the method disclosed herein is an anterior ocular disease or disorder.

In another particular embodiment, the ocular disease or disorder treated using the method disclosed herein is a posterior ocular disease or disorder.

In one embodiment, the ocular disease or disorder is a retinal disease or disorder, In a particular embodiment, the retinal disease or disorder is age-related macular degeneration (AMD) or diabetic retinopathy (DR).

In another embodiment, the ocular disease or disorder is a corneal disease or disorder. The cornea, the transparent and avascular tissue of the anterior ocular segment, is the major refractive surface of the eye, as well as a protective barrier to physical and pathogenic injury. In a particular embodiment, the corneal disease or disorder is an injury (e.g., burn, abrasion), infection (e.g., viral, bacterial, fungal), allergy, dry-eye, corneal edema, corneal degeneration or a heritable corner disease (e.g., a cornea dystrophy).

In a particular embodiment, the ocular disease or disorder is a primary disorder. In another particular embodiment, the ocular disease or disorder is associated with an underlying disease or disorder, such as a chronic disorder (e.g., diabetes) or an acute disorder (e.g., bacterial infection or viral). In another particular embodiment, the ocular disease or disorder is associated with injury or trauma (e.g., chemical burn, foreign body).

In a particular embodiment, the disease or disorder is associated with ocular inflammation for example, uveitis, dry eye disease, keratitis, allergic eye disease, infectious keratitis, herpetic keratitis, retinitis, choroiditis, Behcet's disease, wet and dry age-related macular degeneration (ARMD), and the like.

In another particular embodiment, the disease or disorder is associated with ocular neovascularization.

Non-limiting ocular disorders include, for example, AMD (e.g., wet AMD, dry AMD, intermediate AMD, advanced AMD, and geographic atrophy (GA)), macular degeneration, macular edema, DME (e.g., focal, non-center DME and diffuse, center-involved DME), retinopathy, diabetic retinopathy (DR) (e.g., proliferative DR (PDR), non-proliferative DR (NPDR), and high-altitude DR), other ischemia-related retinopathies, ROP, retinal vein occlusion (RVO) (e.g., central (CRVO) and branched (BRVO) forms), diabetic neuropathy, CNV (e.g., myopic CNV), corneal neovascularization, diseases associated with corneal neovascularization, retinal neovascularization, diseases associated with retinal/choroidal neovascularization, pathologic myopia, von Hippel-Lindau disease, histoplasmosis of the eye, FEVR, Coats' disease, Norrie Disease, OPPG, subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis (e.g., infectious conjunctivitis and non-infectious (e.g., allergic) conjunctivitis), Leber congenital amaurosis (also known as Leber's congenital amaurosis or LCA), uveitis (including infectious and non-infectious uveitis), choroiditis (e.g., multifocal choroiditis), ocular histoplasmosis, blepharitis, dry eye, traumatic eye injury, Sjogren's disease, and other ophthalmic diseases wherein the disease or disorder is associated with ocular neovascularization, vascular leakage, and/or retinal edema. Additional exemplary ocular disorders include diseases associated with rubeosis (neovascularization of the angle) and diseases caused by the abnormal proliferation of fibrovascular or fibrous tissue, including all forms of proliferative vitreoretinopathy.

Dosages and dosing frequency will be determined by a trained medical professional depending on the activity of the compounds used, the characteristics of the particular topical formulation, and the identity and severity of the ocular disorder treated or prevented.

In one embodiment, the nanoformulation is applied once or twice per day or more, depending on the severity of the condition.

In another embodiment, the nanoformulation is applied once or twice per week or more, depending on the severity of the condition.

In one embodiment, the in vitro penetration of the CBD nanoformulation disclosed herein is improved relative to conventional CBD formulations. Various in vitro models of ocular administration are known in the art, such as rabbit and mice models.

In a particular embodiment, the in vitro penetration of the CBD nanoformulation disclosed herein is improved by about 5% to about 50%, compared to a conventional CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%.

In another embodiment, the in vivo penetration of the CBD nanoformulation disclosed herein is improved relative to conventional CBD formulations. The in vivo penetration may be in an animal (e.g., mouse) model or a human patient.

In a particular embodiment, the in vivo penetration of the CBD nanoformulation disclosed herein is improved by about 5% to about 50%, compared to a conventional CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%.

In a particular embodiment, the CBD nanoformulation disclosed herein has an increased permeability coefficient, kip, relative to a conventional CBD formulation. In a particular embodiment, the permeability coefficient is increased by about 50% or more. In a particular embodiment, the permeability coefficient is about 1.0 times, about 1.5 times, about 2.0 times, about 2.5 times, about 3.0 times or about 3.5 times compared to a conventional CBD formulation.

In a particular embodiment, the CBD nanoformulation disclosed herein has improved corneal permeability compared to conventional CBD formulations. In one embodiment, the corneal permeability is improved by about 5% to about 50%, compared to a conventional CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%. Corneal permeability may be measured, for example, using excised rabbit corneas.

In another embodiment, the CBD nanoformulation has a bioavailability of greater than about 10%, greater than about 15%, greater than about 20%, greater than about 25%, greater than about 30%, greater than about 35%, greater than about 40%, greater than about 45% or greater than about 50% or more.

In a particular embodiment, the CBD nanoformulation has a bioavailability of between about 15% and about 50%, about 20% and about 45%, about 25% and about 40% or about 30%.

In another embodiment, the CBD nanoformulation disclosed an improved bioavailability of about 5% to about 50%, compared to a conventional topical CBD formulation, for example, by about 5% to about 50%, by about 5% to about 40%, by about 5% to about 30%, by about 5% to about 20%, by about 5% to about 10%, by about 10% to about 50%, by about 10% to about 40%, by about 10% to about 30%, by about 10% to about 20%, by about 20% to about 50%, by about 20% to about 40%, by about 20% to about 30%, by about 30% to about 50%, by about 30% to about 40%, or by about 40% to about 50%.

In a particular embodiment, the CBD nanformulation disclosed herein has an increase in maximum steady state flux, Jmax, relative to a conventional topical CBD formulation. In a particular embodiment, the maximum steady state flux is increased by about 25% or more.

In another embodiment, the CBD nanoformulation disclosed herein has improved mucoadhesive properties compared to formulations known in the art.

In a particular embodiment, the CBD nanoformulation disclosed herein has an increased mean residence time (MRT) in the eye compared to formulations known in the art. When measured by fluorescence signal, the CBD formulation disclosed herein has a signal intensity that is about 5%, about 10%, about 15%, about 20% or about 25% or more than formulations known in the art. In a particular embodiment, the residence time is percorneal residence time.

In on embodiment, the method disclosed herein reduces one or more symptoms of the ocular disease or disorder. As used herein the term “reducing” refers to a statistically significant and measurable reduction in activity relative to a control. In a particular embodiment, the reduction relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

The signs or symptoms to be monitored will be characteristic of the ocular disorder (e.g., glaucoma) and will be well known to the skilled clinician, as will the methods for monitoring the signs and conditions. In one embodiment, the signs and symptoms associated with an ocular disorder (e.g., glaucoma) may be monitored by assessment via a visual acuity (VA) test. The visual acuity test is used to determine the smallest letters a person can read on a standardized chart or card held 14-20 feet away. This test is done on each eye, one at a time. If necessary, it is then repeated while the subject wears glasses or contacts.

In a particular embodiment, the one or more symptoms are selected from discomfort irritation, burning, itch, grit, redness, inflammation, eye fatigue, bulging, dark spots, distorted vision (e.g., cloudy vision, double vision), flashes of light, light sensitivity, halos, vision loss (e.g., central, general, peripheral), abnormal ocular movement, discharge, pain, nausea, vomiting or headache.

In on embodiment, the method disclosed herein permits treatment while reduces one or side effects compared to conventional or known CBD formulations. In a particular embodiment, the reduction in side effects relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

The side effect may be any reported side effect, for example, ocular discomfort, visual disturbances, infections, headaches, nausea, dizziness, fatigue or the like. In one embodiment, the nanoformulation disclosed herein is less irritating than formulations known in the art.

In a particular embodiment, the CBD nanoformulations disclosed herein have reduced toxicity compared to conventional CBD formulations. Toxicity can be measured, for example, in animal models (e.g., excised rabbit eyes) by gross or microscopic observation of pathology as well as and histopathological analysis. Alternatively, toxicity can be measured by increases in one or more markers of inflammation.

In certain embodiments, the composition disclosed herein may be co-administered with one or more additional active agents. In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The one or more additional active agents may be known to be useful in ocular disease, or may be adjunctive agents that may not be effective alone, but may contribute to the efficacy of the active agent.

The additional active agents may be, for example, anesthetics, analgesics, anti- allergenics, antihistamines, anti-inflammatory agents, anti-cancer agents, antibiotics, antiinfectives, antibacterials, anti-fungal agents, anti-viral agents, cell transport/mobility impending agents, antiglaucoma drugs, mucomimetics, mucogenics, secretagogues, demulcents, wetting agents, lubricants, hypertensives, decongestants, immunological response modifiers, immunosuppresive agents, peptides, proteins, steroidal compounds, steroids, low solubility steroids, carbonic anhydrize inhibitors, diagnostic agents, antiapoptosis agents, gene therapy agents, sequestering agents, reductants, antipermeability agents, antisense compounds, antiproliferative agents, antibodies, antibody conjugates, bloodflow enhancers, antiparasitic agents, non-steroidal anti inflammatory agents, nutrients, vitamins, enzyme inhibitors, antioxidants, anticataract drugs, aldose reductase inhibitors, cytoprotectants, cytokines, cytokine inhibitors, cytokine protectants, UV blockers, mast cell stabilizers, anti-neovascular agents, antiangiogenic agents, matrix metalloprotease inhibitors, vascular endothelial growth factor (VEGF) modulators, neuroprotectants, miotics, anti-cholinesterase, mydriatics and ocular lubricants and artificial tear/dry eye therapies.

In certain embodiments, the method can also include administering an anesthetic (e.g. lidocaine) to the eye concurrently with or prior to nanoformulation disclosed herein.

### IV. Method of Manufacture

Cannabinoids exhibit poor water solubility, which complicates their delivery to the blood stream and reduces the associated bioavailability. Particle size reduction to the nanometer range of these substances increases their aqueous dissolution rate and solubility, which results in improved bioavailability, accelerated onset of action, and decreased potential of harmful side-effects.

The lipid-based nanoparticles disclosed herein may be produced by any suitable method. In one embodiment, the lipid-based nanoparticle is produced by a method selected from top down methods, bottom up methods or a combination thereof.

In a particular embodiment, the lipid-based nanoparticle is prepared by a method selected from high pressure homogenization (e.g., hot or cold high-pressure homogenization), double emulsion, microemulsion, ultrasonication, solvent evaporation, solvent emulsification-diffusion, super critical fluid methods, spray drying or combinations thereof.

In a particular embodiment, the lipid-based nanoparticle is a liposome and the liposome is produced by dissolving the cannabidiol in an organic solvent, then mixing the same with lipids dissolved in a miscible organic solvent. The thin lipid film produced by rotary evaporation is then hydrated by adding an aqueous solution. The resultant multilamellar liposomes are extruded through membranes with defined pore size or sonicated to form small unilamellar vesicles of desired size.

In a particular embodiment, the lipid-based nanoparticle is made from high pressure homogenization (HPH). The term “homogenization” refers to the production of a homogeneous size distribution of particles suspended in a liquid, by forcing the liquid under the effect of pressure through a specifically designed homogenization valve. According to this technique, the cannabidiol is first solubilized in the melted lipid.

In another particular embodiment, the lipid-based nanoparticle is made by ultrasonication. In a particular embodiment, the method is probe ultrasonification (i.e., direct or indirect). The process of ultrasonic top-down nanocrystallization requires extremely high ultrasonic amplitudes to be applied to particle suspensions producing extreme shear forces. The shear forces are the result of intense ultrasonic cavitation, which creates imploding vacuum bubbles and causes micro-jets that break up the original drug particles down to the nano-size range.

The ultrasonication settings comprise one or more of an amplitude, a frequency, a power, and a duration. For example, in some embodiments, the ultrasonication setting includes an amplitude between 25 and 100 microns. In certain embodiments, the ultrasonication is applied at a frequency in the range of 20 Hz to 20 kHz. In some embodiments, the ultrasonication is applied at a frequency around 200 Hz, e.g., in the range of 175 to 225 Hz. In some embodiments, the ultrasonication applied at a power in the range from 100 to 400 W. In some embodiments, the ultrasonication is applied for a particular duration, such as for 15 minutes or longer.

In a particular embodiment, ultrasonic amplitudes of at least 70 microns are used to take full advantage of ultrasonic cavitation.

In another embodiment, the nanoparticles are made to an average size of 50 nms for enhanced penetration and bioavailability.

In certain embodiments, the lipid-based nanoparticle composition comprises cannabidiol that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have cannabidiol in them.

Characteristics such as loading capacity, drug release rate, physical and chemical stability, and vesicle size depend on experimental conditions, and material and method choices at the time of preparation, as would be understood by one of skill in the art.

### EXAMPLES

### Example 1:

A fractionated coconut oil is provided containing caproic acid (C6) and caprylic (C8) acid, but substantially no fatty acids having more than 8 carbon atoms.

The fractionated coconut oil is used to form a lipid-based nanoparticle.

The lipid-based nanoparticle is then loaded with cannabidiol to provide a nanoparticle formulation.

The nanoparticle formulation is shown to have greater than about 15% bioavailability by an in vitro assay.

The nanoparticle formulation is shown to have greater than about 15% bioavailability by an in vivo assay.

1. A method of treating an ocular disease or disorder comprising administering an effective amount of a formulation comprising a lipid-based nanoparticle to a subject in need therein, wherein the lipid-based nanoparticle comprises (i) cannabidiol and (ii) at least one fractionated oil, wherein the fractionated oil comprises at least one short-chain fatty acids having between 6 and 8 carbon items and does not contain any fatty acids having greater than 8 carbon atoms.

2. The method of claim 1, wherein the at least one fractionated oil is coconut oil.

3. The method of claim 1, wherein the lipid-based nanoparticle is suitable to provide for increased ocular permeability and bioavailability of the cannabidiol.

4. The method of claim 1, wherein the lipid-based nanoparticle further comprises at least one additional cannabinoid.

5. The method of claim 1, wherein the lipid-based nanoparticle is in the form of a liposome or lipid-drug conjugate.

6. The method of claim 1, wherein the lipid-based nanoparticle is in the form of a solid lipid nanoparticle (SLN).

7. The method of claim 1, wherein the lipid-based nanoparticle is in the form of a nanostructured lipid particle (NLP).

8. The method of claim 1, wherein the lipid-based nanoparticle is about 50 nms.

9. The method of claim 1, wherein the ocular disease or disorder is an anterior ocular disease or disorder.

10. The method of claim 1, wherein the ocular disease or disorder is a posterior ocular disease or disorder.
